(19) United States
(12) Patent Application Publication
(10) Pub. No.: US 2005/0240095 A1
(43) Pub. Date: Oct. 27, 2005

(54) Variable flip angle, sar-reduced state free precession mri acquisition

(75) Inventor: Tobias Schaffter, Hamburg (DE)

Correspondence Address: Thomas M Lundin;Philips Intellectual Property & Standards, 595 Miner Road, cleveland, OH, 44143, US

(73) Assignee: KONINKLIJKE PHILIPS ELECTRONICS N.V., (omitted)

(21) Appl. No.: 10/513,209

(22) PCT Filed: Apr. 29, 2003
(86) PCT No.: PCT/IB03/01635

§ 371 Date: Nov. 2, 2004

(30) Foreign Application Priority Data
May 2, 2002 (DE) 102-19-528.5

(51) Int. Cl. G01V 3/00; A61B 5/05

(52) U.S. Cl. 600/410; 324/309

(57) ABSTRACT

The invention relates to a magnetic resonance imaging method in which gradient echo signals (E1, E2, E3) are repeatedly acquired for a plurality of phase encoding values. In order to reduce the RF load whereto a patient to be examined is exposed, the flip angles of RF excitation pulses (HF1, HF2, HF3, HF4) of the pulse sequence are varied in dependence on the phase encoding value. For optimization of the image contrast it is advantageous when the flip angle is maximum for minimum absolute phase encoding values and minimum for maximum absolute phase encoding values.

The invention relates to a magnetic resonance imaging method for imaging at least a part of a body, which is situated in a steady and essentially homogeneous main magnetic field, which method includes the steps of:
exciting nuclear magnetization in the body in the direction transversely of the main magnetic field direction by application of an RF excitation pulse with which a selectable flip angle of the nuclear magnetization is associated,
phase encoding the transverse nuclear magnetization in conformity with a phase encoding value by generating at least one magnetic field gradient pulse of corresponding duration and amplitude in a phase encoding direction,
generating at least one gradient echo signal of the transverse nuclear magnetization by temporally successively generating at least one dephasing magnetic field gradient pulse and at least one rephasing magnetic field gradient pulse in a read-out direction,
measuring the gradient echo signal,
acquiring a set of gradient echo signals by repeating the steps a) to d) after a repetition time for a plurality of different phase encoding values,
transforming the set of gradient echo signals to an image of the body.

The invention also relates to an apparatus for magnetic resonance imaging in conformity with such a method.

In addition to the steady and essentially homogeneous main magnetic field, pulse sequences consisting of RF pulses and magnetic field gradient pulses act on the body of a patient to be examined by means of the known MRI methods. As a result, in the body of the patient there are generated magnetic resonance signals which are detected by means of suitable receiving devices (antennas or coils) of a magnetic resonance apparatus. Conventionally a Fourier transformation is applied to this data so as to reconstruct an image of the body of the patient, which is suitable for diagnostic purposes.

The number of clinically relevant fields of application of MRI has increased enormously in recent times. The method can be used for the examination of practically every part of the human body; it is notably also possible to study important body functions such as the transport of blood, the cardiac cycle or the respiration. The scanning of the so-called k-space is governed by the number, the spacing in time, the duration and the strength of the RF pulses and magnetic field gradient pulses used, so that the relevant pulse sequence used completely determines the properties of the reconstructed image, such as the position and orientation of the part of the body being examined, the dimensions of the selected image detail, the resolution, the signal-to-noise ratio, the contrast, the sensitivity to motions etc.

One of the essential problems encountered in magnetic resonance imaging consists in that the acquisition of a complete image of a quality, which suffices for diagnostic purposes, usually requires an undesirably long period of time. Notably in the field of functional and interventional magnetic resonance imaging there is a need for very fast methods enabling the study of dynamic processes within the body of the patient or the execution of surgical interventions with monitoring by magnetic resonance imaging.

The pulse sequence used in the above method is known as a “gradient echo” sequence. This term covers customary sequences which are normally denoted by the abbreviations GRE (Gradient Echo), FFE (Fast Field Echo), GRASS (Gradient Recalled Acquisition in the Steady State), FISP (Fast Imaging with Steady-state Precession; see, for example, Oppelt et al. in “Electromedica”, issue 54, No. 1, 1986, pp. 15 to 18) or also EPI (Echo Planar Imaging). These pulse sequences are characterized by a particularly short image acquisition time because, unlike the equally customary so-called Spin Echo methods, they do not utilize time consuming RF pulses (180° pulses) for the refocusing of the nuclear magnetization. In the above method, the gradient echo is generated exclusively, without application of refocusing RF pulses in the step c) of the method, in that first a dephasing magnetic field gradient pulse and subsequently a rephasing magnetic field gradient pulse is generated in the read-out direction. During the measurement of the gradient echo signal in the step d) of the method, the magnetic field gradient is sustained in the read-out direction for the purpose of frequency encoding, so that the nuclear magnetization dephases again.

The very fast gradient echo methods have proven their worth inter alia for dynamic cardiac studies, for magnetic resonance angiography and also for the examination of articular cartilage. It is particularly advantageous that the described gradient echo method is equally suitable for two-dimensional as well as three-dimensional imaging when phase encoding is carried out in one and in two spatial directions, respectively, in the step b) of the method.

For as fast as possible image acquisition, the pulse sequence in the gradient echo method is carried out with as short as possible repetition times. However, the patient to be examined is then exposed to the RF excitation pulse in the step a) of the method in rapid succession. This RF exposure causes heating of the body tissue, so that in the case of fast imaging there is a risk of the physiologically acceptable limit being exceeded for the patient. Therefore, the clinical use of magnetic resonance imaging is subject to rules defining the maximum amount of RF energy that may be applied per unit of time (so-called Specific Absorption Rate or SAR).

In order to circumvent this problem, it is not possible to use general RF excitation pulses with an as small as possible amplitude and duration so as to minimize the applied RF power. This is because it is known that in gradient echo methods the image contrast is rather dependent on the flip angle of the RF excitation pulse.

Considering the foregoing, it is an object of the present invention to provide a gradient echo method, which enables an extremely short image acquisition time in combination with a minimum RF load for the patient.

On the basis of a method of the kind set forth, this object is achieved in that the flip angle of the RF excitation pulse is varied in dependence on the relevant phase encoding value during the acquisition of the set of gradient echo signals.

In order to reduce the RF load in accordance with the invention, the amplitude or the duration of the RF excitation pulse, and hence the flip angle, is deliberately reduced for those phase encoding values which are of little importance for the image contrast. It has been found that the RF power whereto the body of the patient to be examined is exposed is inversely proportional to the repetition time and directly proportional to the square of the flip angle of the RF excitation pulse. Therefore, in accordance with the invention a considerable acceleration of the image acquisition can be achieved already by way of a small reduction of the flip angle.

Granted, from U.S. Pat. No. 5,704,357 it is known to reduce the amplitude of the 180° refocusing pulses for large phase encoding values in a fast spin echo method so as to reduce the RF load. The method in conformity with the present invention, however, is a gradient echo method, which already operates completely without refocusing pulses, so that said United States patent is not effective in achieving the object of the invention. Until now there was a widespread notion that for gradient echo methods it is not necessary at all to reduce the RF power further because, instead of the customarily large number of 180° refocusing pulses in the spin echo method, only comparatively few excitation pulses with substantially smaller flip angles are used (see Oppelt et al. as cited above).

The invention is based on the idea that in order to achieve an optimum image contrast for gradient echo methods it is not necessary to keep the flip angle of the RF excitation pulse constant during the entire image acquisition if a particularly short image acquisition time is desired.

In conformity with the method of the invention, during the acquisition of the set of gradient echo signals the flip angle is advantageously varied in such a manner that it assumes a maximum value when the absolute value of the phase encoding value is minimum, and that it assumes a minimum value other than zero when the absolute value of the phase encoding value is maximum during the acquisition of the set of gradient echo signals. According to this procedure the center of the k-space, being decisive for the image contrast, is scanned with a maximum flip angle while the outer regions of the k-space, being less important for the image contrast, are scanned with a minimum flip angle, so that the resultant reduction of the RF load for the patient is achieved at the expense of only an insignificant effect on the image quality.

In order to minimize the RF load, for the method in accordance with the invention it makes sense to vary the flip angle in steps between the minimum value and the maximum value in dependence on the phase encoding value. The flip angle of the RF excitation pulse thus decreases in steps in the direction from the center of the k space to the outer regions thereof. Notably in the case of gradient echo methods, operating with a dynamic steady state of the nuclear magnetization (so-called Steady State methods such as, for example, GRASS or FISP), the variation of the flip angle in accordance with the invention causes overshoot or undershoot of the amplitude of the echo signal from one phase encoding value to another phase encoding value. Because of the step-wise, that is, gradual, variation of the flip angle, excessive disturbances of the dynamic steady state of the nuclear magnetization are avoided. Such disturbances could otherwise give rise to undesirable image artifacts.

It has been found that for the method in accordance with the invention it is advantageous to perform the acquisition of the set of gradient echo signals for a plurality of equidistant phase encoding values, which are ordered in conformity with their absolute value. For the image acquisition the phase encoding values are thus ordered in such a manner that the k-space is scanned from a minimum absolute phase encoding value (kₘᵢₙ) to a maximum absolute phase encoding value (kₘₐₓ). As a result, the disturbances of the dynamic steady state of the nuclear magnetization in steady state methods, caused by the variation of the flip angle, can be predicted and controlled better. In this respect it is important that the absolute phase encoding value varies only slowly in the course of the image acquisition.

Because the flip angle of the RF excitation pulse is determined by a continuous function of the phase encoding value in the method in accordance with the invention, the image contrast can be optimized in a particularly advantageous manner while at the same time the RF load is minimized. The functional dependency of the flip angle on the phase encoding value can then be adapted to the relevant application by way of a few parameters.

Special advantages are obtained when in the method in accordance with the invention the application of the RF excitation pulse in the step a) takes place alternately with an alternating phase and when, after each measurement of the gradient echo signal in the step d) of the method and before the application of the next RF excitation pulse in the subsequent step a) of the method, each time at least one magnetic field gradient pulse is generated in the phase encoding direction and in the read-out direction in such a manner that the effect of the magnetic field gradient pulses generated in the steps b) and c) of the method on the phase of the transverse nuclear magnetization is compensated. This actually concerns a further elaboration of the known gradient echo method, which utilizes a dynamic steady state of the nuclear magnetization during the image acquisition (for example, GRASS, FISP, see above). As a result of this approach it is achieved that the nuclear magnetization remaining after each measurement in the step d) of the method contributes to the echo signal during the respective next repetition of the steps a) to d) of the method. As a result, the signal amplitude, the signal-to-noise ratio and ultimately the image contrast are optimized with a minimum image acquisition time. In these steady-state methods the amplitude of the echo signal is highly dependent on the flip angle of the RF excitation pulse. For example, in the case of the FISP sequence it is necessary to use comparatively large flip angles in order to ensure that the signal amplitude is adequate. Therefore, pulse sequences of this kind are particularly problematic in respect of the RF load for the patient, so that it is advantageous to vary the flip angle of the RF excitation pulse in dependence on the phase encoding value in accordance with the invention.

The effect of the variation of the flip angle on the amplitude of the gradient echo signal can be advantageously compensated in the method in accordance with the invention by weighting the measured gradient echo signals with a corresponding function prior to the transformation in the step f) of the method. The theoretical knowledge of the functional relationship between flip angle and signal amplitude can thus be used to avoid undesirable image artefacts as caused by the variation of the flip angle. The weighting function to be used is dependent not only on the value of the flip angle of the RF excitation pulse, but also on the nuclear magnetization relaxation times T₁ and T₂ which, however, are known in most cases.

An MRI apparatus as disclosed in the claims 8, 9 and 10 is suitable for carrying out the method in accordance with the invention. A conventional apparatus in clinical use can be advantageously adapted in conformity with the invention merely by programming the control and reconstruction means accordingly. The software required for this purpose can be advantageously made available to the users of magnetic resonance imaging apparatus on a suitable data carrier, such as a disc or a CD-ROM, or by downloading via a data network (the Internet).

Embodiments of the invention will be described in detail hereinafter with reference to the Figs. Therein:

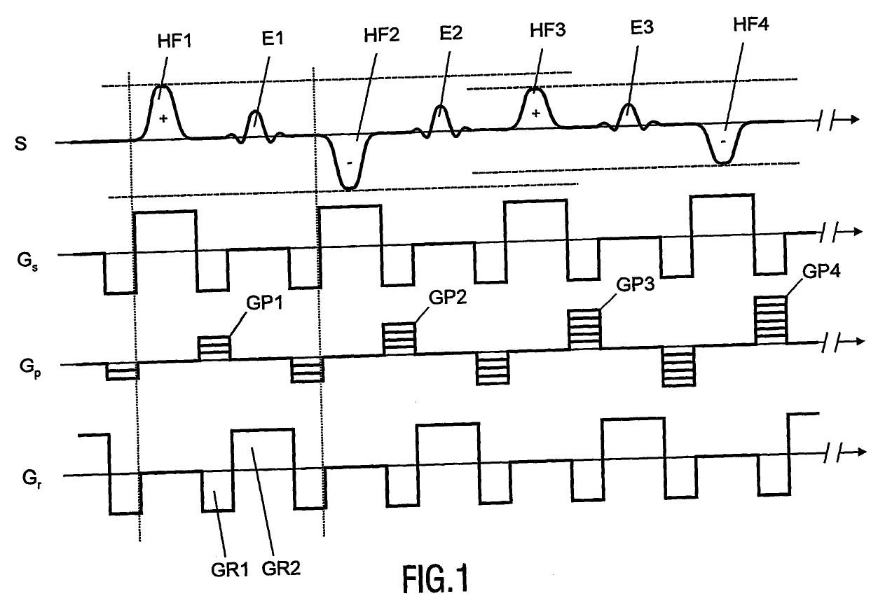

FIG. 1 shows a flow chart of the method in accordance with the invention,

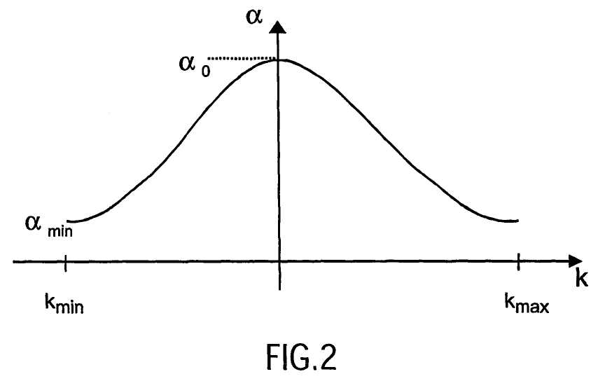

FIG. 2 shows a diagram illustrating the functional dependency of the flip angle on the phase encoding value, and

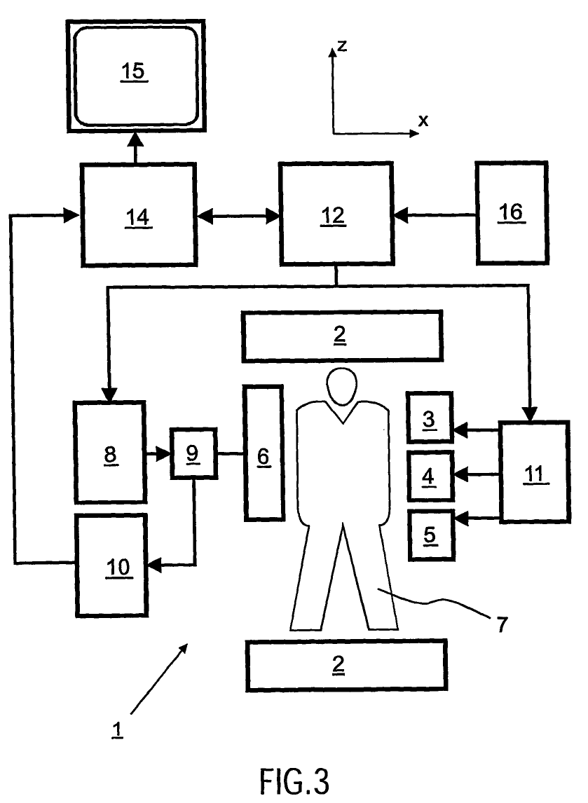

FIG. 3 is a diagrammatic representation of a magnetic resonance imaging apparatus in accordance with the invention.

The uppermost time-dependency diagram, denoted by the reference S in

FIG. 1, shows RF excitation pulses HF1, HF2, HF3 and HF4. The diagram therebelow shows the variation in time of a magnetic field gradient Gₛ which is used for slice selection. The third diagram shows pulses, generated in a phase encoding direction, of a magnetic field gradient Gₚ. The lowermost diagram shows the variation in time of a read-out gradient Gr. The gradients Gₛ, Gₚ and Gᵣ extend in mutually perpendicular spatial directions. The pulse sequence shown in FIG. 1 corresponds to a FISP sequence, which has been modified in conformity with the invention. As described above, this sequence is a gradient echo sequence in which the nuclear magnetization is in a dynamic steady state. FIG. 1 shows only a part of the sequence continuously applied during the image acquisition. A first cycle of the method shown consists of the RF and magnetic field gradient pulses, which are generated in the time interval defined by the vertical dotted lines. This interval commences with the application of the RF excitation pulse HF1 (step a) of the method). During the application of the pulse BF1, the slice selection gradient Gs is also active, so that the transverse nuclear magnetization is excited in a dedicated manner in a predetermined slice of the body of the patient. The image plane of the image to be formed is thus determined. The phase encoding of the transverse nuclear magnetization is performed by means of a magnetic field gradient pulse GP1 (step b) of the method). The amplitude thereof determines the associated phase encoding value keg. During the next step (step c) of the method) a magnetic field gradient pulse GR1, causing dephasing of the transverse nuclear magnetization, and a pulse GR2, having a polarity which opposes that of the pulse GR1, are generated successively in time, thus causing rephasing. This results in a gradient echo signal E1 which is measured while the read-out gradient GR2 is sustained (step d) of the method). This cycle is succeeded by further cycles in which gradient echo signals E2 and E3 are measured. The phase of the RF excitation pulses HF2 and HF4 then opposes that of the pulses HF1 and HF3. The alternating phase leads to maximization of the usable nuclear magnetization. Overall the steps a) to d) are repeated until a set of gradient echo signals has been measured wherefrom an image of the body of the patient can be reconstructed. It can be recognized in the diagram that the variation in time of each of the gradients Gₛ, Gₚ and Gᵣ during a cycle is predetermined in such a manner that the overall effects of the gradient pulses on the nuclear magnetization compensate one another. It is thus achieved that the nuclear magnetization remaining after the measurement of each gradient echo signal E1, E2 and E3 again contributes to the signal during the respective subsequent measurement. Such gradient echo methods are also known as “Balanced Fast Field Echo” (Balanced FFE) methods. Gradient pulses GP2, GP3 and GP4 determine phase encoding values k₂, k₃ and k₄ for the further cycles shown in the diagram. In conformity with the invention, the amplitude of the RF excitation pulses HF3 and HF4 is reduced for the larger phase encoding values k₃ and k₄ in order to reduce the RF load for the patient, that is, in comparison with the pulses HF1 and HF2, respectively. Consequently, the signal amplitude of the gradient echo E3 is smaller than for the preceding measurements. The image contrast, however, is only hardly affected thereby, because it is governed more strongly by the amplitudes of echo signals associated with smaller k values.

The diagram of FIG. 2 shows a feasible functional dependency of the flip angle α of the RF excitation pulse on the phase encoding value k in accordance with the invention. For the minimum absolute phase encoding value k=0 the function assigns a maximum value α₀ to the flip angle. For the maximum absolute phase encoding values kₘᵢₙ and kₘₐₓ the flip angle assumes a minimum value αₘᵢₙ other than zero. The function shown is a Gaussian function whose parameters enables optimum adjustment of the RF load on the one hand and the image contrast on the other hand.

FIG. 3 is a diagrammatic representation of an apparatus for magnetic resonance imaging in accordance with the invention. The apparatus 1 consists of a main field magnet 2 for generating a steady, essentially homogeneous main magnetic field. Three gradient coils 3, 4 and 5 serve to generate gradient magnetic fields which are superposed on the main magnetic field and have a respective, adjustable strength in different spatial directions. The direction of the main magnetic field is by convention referred to as the z direction and the two directions perpendicular thereto as the x direction and the y direction, respectively. The gradient coils 3, 4 and 5 receive a current from a gradient amplifier 11. The apparatus 1 also includes a transmission device 6, being an antenna or a coil, for applying RF pulses to an examination volume of the apparatus which is traversed by the main magnetic field and in which a patient 7 is arranged. The transmission device 6 is connected to a modulator 8 so as to generate the RF pulses. Furthermore, there is provided a receiving device for receiving magnetic resonance signals from the examination volume. In the apparatus shown in FIG. 3 the transmission device and the receiving device are formed by the same antenna or coil. Therefore, a switch 9 is required for switching over between the transmission mode and the receiving mode. The magnetic resonance signals received are applied to a demodulator 10. The modulator 8, the transmission device 6 and the gradient amplifier 11 are controlled by a control device 12 so as to generate the described pulse sequence in accordance with the invention. The control device is a microcomputer which includes a memory and program control means. For a practical implementation of the invention the memory contains a program with a description of the imaging pulse sequence in conformity with the method of the invention. The demodulator 10 is connected to a reconstruction unit 14 which is also a computer. This unit transforms the set of echo signals received into an image which is displayed on a display screen 15.

1. A magnetic resonance imaging method for imaging at least a part of a body which is situated in a steady and essentially homogeneous main magnetic field, which method includes the steps of:
exciting nuclear magnetization in the body in the direction transversely of the main magnetic field direction by application of an RF excitation pulse with which a selectable flip angle of the nuclear magnetization is associated,
phase encoding the transverse nuclear magnetization in conformity with a phase encoding value by generating at least one magnetic field gradient pulse of corresponding duration and amplitude in a phase encoding direction,
generating at least one gradient echo signal of the transverse nuclear magnetization by temporally successively generating at least one dephasing magnetic field gradient pulse and at least one rephasing magnetic field gradient pulse in a read-out direction,
measuring the gradient echo signal,
acquiring a set of gradient echo signals by repeating the steps a) to d) after a repetition time for a plurality of different phase encoding values,
transforming the set of gradient echo signals into an image of the body, wherein the flip angle of the RF excitation pulse is varied in dependence on the relevant phase encoding value during the acquisition of the set of gradient echo signals.

2. A method as claimed in claim 1, wherein during the acquisition of the set of gradient echo signals the flip angle is varied in such a manner that it assumes a maximum value when the absolute value of the phase encoding valued is minimum, and that it assumes a minimum value other than zero when the absolute value of the phase encoding value is maximum during the acquisition of the set of gradient echo signals.

3. A method as claimed in claim 2, wherein the flip angle is varied in steps between the minimum and the maximum value in dependence on the phase encoding value.

4. A method as claimed in claim 1, wherein the acquisition of the set of gradient echo signals is performed for a plurality of equidistant phase encoding values which are ordered in conformity with their absolute value.

5. A method as claimed in claim 1, wherein the flip angle is determined by a continuous function of the phase encoding value.

6. A method as claimed in claim 1, wherein the application of the RF excitation pulse in the step a) takes place alternately with an alternating phase and that, after each measurement of the gradient echo signal in the step d) of the method and before the application of the next RF excitation pulse in the subsequent step a) of the method, each time at least one magnetic field gradient pulse is generated in the phase encoding direction and in the read-out direction in such a manner that the effect of the magnetic field gradient pulses generated in the steps b) and c) of the method on the phase of the transverse nuclear magnetization is compensated.

7. A method as claimed in claim 1, wherein the effect of the variation of the flip angle on the amplitude of the gradient echo signal is compensated in that the measured gradient echo signals are weighted with a corresponding function prior to the transformation in the step f) of the method.

8. An apparatus for magnetic resonance imaging of at least a part of a body which is situated in a steady and essentially homogeneous main magnetic field in conformity with a method as claimed in one of the preceding claims, which apparatus includes means for generating the main magnetic field, means for generating gradient magnetic fields which are superposed on the main magnetic field, means for applying RF pulses to the body, control means for controlling the means for generating the gradient magnetic fields and the means applying the RF pulses, means for receiving and acquiring magnetic resonance signals, and reconstruction means for transforming the acquired magnetic resonance signals into an image of the body, the control means and the reconstruction means being programmed in such a manner that the following steps of the method can be carried out thereby:
applying an RF excitation pulse which is associated with a selectable flip angle of the nuclear magnetization,
generating at least one magnetic field gradient pulse in a phase encoding direction, the duration and/or amplitude of said pulse corresponding to a phase encoding value,
temporally successively generating at least one dephasing magnetic field gradient pulse and at least one rephasing magnetic field gradient pulse in a read-out direction,
measuring at least one gradient echo signal,
acquiring a set of gradient echo signals by repeating the steps a) to d) after a repetition time for a plurality of different phase encoding values,
transforming the set of gradient echo signals into an image of the body, wherein the control means are also programmed in such a manner that the flip angle of the RF excitation pulse is varied in dependence on the relevant phase encoding value during the acquisition of the set of gradient echo signals.

9. An apparatus as claimed in claim 8, wherein the control means determine the flip angle by way of a function of the phase encoding value, the function assigning a maximum flip angle to a minimum absolute phase encoding value and a minimum flip angle other than zero to a maximum absolute phase encoding value.

10. An apparatus as claimed in claim 8, wherein the control means are programmed in such a manner that the application of the RF excitation pulse in the step a) of the method takes place alternately with an alternating phase and that, after each measurement of the gradient echo signal in the step d) of the method and before the application of the next RF excitation pulse in the subsequent step a) of the method, each time at least one magnetic field gradient pulse is generated in the phase encoding direction and in the read-out direction in such a manner that the effect of the magnetic field gradient pulses generated in the steps b) and c) of the method on the phase of the transverse nuclear magnetization is compensated.
